(19) United States
(12) Patent Application Publication
(10) Pub. No.: US 2026/0191442 A1
(43) Pub. Date: Jul. 9, 2026

(54) Eccentric Single-Core Fiber-Optic Enabled Medical Device

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 19/554,612

(22) Filed: Mar. 2, 2026

Related U.S. Application Data
(63) Continuation of application No. 17/863,211, filed on Jul. 12, 2022, now Pat. No. 12,564,340.

(51) Int. Cl. A61B 5/1459; A61B 1/00; A61B 1/06; A61B 1/07; A61B 1/12; A61B 1/313; A61B 5/00; A61B 5/0205; A61B 5/026; A61B 5/145; A61B 5/1455; A61B 34/20

(52) U.S. Cl. CPC A61B 5/1459; A61B 1/00009; A61B 1/00013; A61B 1/00045; A61B 1/00055; A61B 1/00057; A61B 1/0661; A61B 1/07; A61B 1/126; A61B 1/3137; A61B 5/02055; A61B 5/0261; A61B 5/14539; A61B 5/14552; A61B 5/6852; A61B 2034/2061; A61B 2562/0271

(57) ABSTRACT

Systems, devices, and methods for performing vascular treatments and diagnoses. A vascular device includes an optical fiber including a single fiber core disposed offset from a central axis of the optical fiber. The single fiber enables logic of the system to determine multiple conditions of the device and the patient. The conditions may include one or more of blood flow parameters, infusate delivery parameters, location of the device within the patient, pH of the blood, oxygen level of the blood, damage to the optical fiber, or core temperature of the patient. The elongate medical device may be a catheter, a stylet, a guidewire, or a probe.

### PRIORITY

This application is a continuation of U.S. patent application Ser. No. 17/863,211, filed Jul. 12, 2022, now U.S. Pat. No. 12,564,340, which is incorporated by reference in its entirety into this application.

### BACKGROUND

Elongate medical devices configured for insertion within a patient vasculature may be utilized to perform a myriad of treatments and diagnoses. Fiber optic enabled vascular devices can be beneficial in the providing the vascular treatments. Fiber optic shape sensing capability can also enable proper placement of the vascular devices. Shapes sensing optical fibers typically utilize multi-core optical fibers. However, multi-core optical fibers result in elevated costs of the medical devices and the related systems. Furthermore, the added complexity of the multi-core optical fibers can negative affect reliability resulting in increased risk to the patient and further increased cost to the healthcare provider.

Disclosed herein are medical systems and methods that address the forgoing.

### SUMMARY

Briefly summarized, disclosed herein is a medical system. According to some embodiments, the medical system includes an elongate medical device configured for insertion within a blood vessel of a patient, where the medical device includes an optical fiber extending along a longitudinal length the medical device to a distal end of the medical device. The optical fiber has a single core fiber extending along the optical fiber, where the single core fiber is disposed radially offset from a central axis of the optical fiber. The single core fiber includes a plurality of sensors distributed along the longitudinal length, where each sensor is configured to (i) reflect a light signal of a different spectral width based on received incident light, and (ii) change a characteristic of the reflected light signal based on a state of the optical fiber.

The system further includes a console operatively coupled with the optical fiber. The console includes a light source, an optical receiver, one or more processors, and a non-transitory computer-readable medium having stored thereon logic that, when executed by the one or more processors, causes operations of the system that include (i) projecting a light distally along the optical fiber, (ii) receiving at least one reflected light signal from the optical fiber, (iii) determining a state of the optical fiber, based on the at least one reflected light signal, and (iv) communicating the state to a user.

In some embodiments of the system, the state of the optical fiber includes a fluctuating movement of at least a distal portion of the optical fiber, and the operations further include (i) extracting from the at least one reflected light signal present fluctuating movement data, (ii) comparing the present fluctuating movement data with one or more fluctuating movement limits stored in the non-transitory computer-readable medium, where the fluctuating movement limits pertain to movement of the distal portion in response to oscillating anatomic motion adjacent a cavoatrial junction of the patient, and (iii) determining, as result of the comparison, that the distal end of the medical device is disposed adjacent the cavoatrial junction the blood vessel.

In some embodiments of the system, the state of the optical fiber further includes a condition experienced by an inserted portion of the optical fiber, and the operations further include (i) receiving one or more reflected light signals from a subset of the plurality of sensors, the subset disposed along the inserted portion, (ii) extracting present condition data from the one or more reflected light signals, and (iii) determining a length of the inserted portion based on the present condition data.

In some embodiments of the system, the state of the optical fiber includes a compressive strain of the optical fiber caused by a contact force applied to the distal end during advancement of the medical device along the blood vessel, and the operations further include (i) extracting from the at least one reflected light signal present compressive strain data, and (ii) identifying from the compressive strain data check valves disposed along the blood vessel.

In some embodiments of the system, the state of the optical fiber includes a compressive strain of the optical fiber caused by a contact force applied to the distal end of the optical fiber, and the operations further include (i) extracting from the at least one reflected light signal present compressive strain data, (ii) comparing the present compressive strain data with a compressive strain safety limit stored in the non-transitory computer-readable medium, the compressive strain safety limit defining a safe limit for contact of the distal end with the anatomical elements, and (iii) providing an alert to the user when the present compressive strain data exceeds the compressive strain safety limit.

In some embodiments of the system, the state of the optical fiber includes a temperature experienced by one or more of the plurality of sensors when the optical fiber is inserted within the patient, and the operations further include (i) receiving one or more reflected light signals from the one or more sensors, (ii) extracting present temperature data from the one or more reflected light signals, and (iii) determining a core temperature of the patient from the present temperature data.

In some embodiments of the system, the optical fiber extends along a catheter inserted within the blood vessel, where the catheter is configured to deliver an infusate to the blood vessel, and the state of the optical fiber includes a first temperature experienced by a section of the optical fiber extending beyond a distal end of the catheter during non-delivery of the infusate and a second temperature experienced by the section during delivery of the infusate. In such embodiments, the operations further include (i) receiving a first reflected light signal from a sensor disposed along the section during non-delivery of the infusate, the first reflected light signal based on the first temperature, (ii) receiving a second reflected light signal from the sensor, the second reflected light signal based on the second temperature, (iii) extracting from the first and second reflected light signals present temperature difference data between the first and second temperatures, (iv) comparing the present temperature difference data with a temperature difference limit stored in the non-transitory computer-readable medium. The operations further include determining, as result of the comparison, (i) when the infusate is delivered and (ii) when the infusate is not delivered.

In some embodiments of the system, the infusate is a flushing solution for the catheter, where initiating and terminating the delivery of the infusate defines a flushing event. In such embodiments, the operations further include determining at least one of a frequency or a number of flushing events.

In some embodiments of the system, the delivery rate of the infusate is known such that initiating and terminating the delivery of the infusate defines an infused volume of the infusate, and the operations further include determining of the infused volume.

In some embodiments of the system, the state of the optical fiber includes a damage to the optical fiber, and the operations further include determining the damage of the optical fiber, where determining the damage includes at least one of (i) receiving a subset of reflected light signals from a respective subset of sensors, the subset of reflected light signals having a spectral width that exceeds a defined spectral-width range stored in the non-transitory computer-readable medium, or (ii) not receiving a reflected light signal from at least one sensor.

In some embodiments of the system, the operations further include (i) determining a longitudinal location of the respective subset of sensors or the at least one sensor along the along the optical fiber and (ii) relating the longitudinal location of the respective subset of sensors or the at least one sensor with a longitudinal location of the damage along the optical fiber.

In some embodiments of the system, the operations further include (i) propagating an illuminating light distally along the optical fiber, (ii) projecting the illuminating light distally away from a distal of the optical fiber, (iii) receiving an image light signal via the distal end, (iv) propagating the image light signal proximally along the optical fiber, (v) extracting image data from the image light signal, and (vi) portraying the image data in the form of an image on a display of the system.

In some embodiments of the system, the optical fiber is inserted within the blood vessel, and the operations further include (i) projecting a light defining a first wavelength distally away from a distal end of the optical fiber into blood of the blood vessel, (ii) receiving a reflected light signal having a second wavelength via the distal end, (iii) extracting from the reflected light signal a present wavelength shift between the first wavelength and the second wavelength, (iv) comparing the present wavelength shift with one or more wavelength shift limits stored in the non-transitory computer-readable medium, and (v) determining, as result of the comparison, at least one of a direction of blood flow or a velocity of the blood flow.

In some embodiments of the system, the operations further include (i) projecting the light toward a coating of the medical device, where the coating is configured to define a light characteristic based on a pH of a fluid in contact with the medical device, (ii) receiving a light signal from the coating in response to the projected light, where the light signal includes the light characteristic, (iii) extracting the pH from the light signal, and (iv) communicating the pH to the user.

In some embodiments of the system, the operations further include (i) projecting the light into blood of the patient, (ii) receiving a light signal from the blood in response to the projected light, where the light signal includes a light characteristic based on a blood oxygen level, (iii) extracting the blood oxygen level from the light signal, and (iv) communicating the blood oxygen level to the user.

In some embodiments of the system, the elongate medical device includes a catheter, a stylet, a probe, or a guidewire.

Also disclosed herein is an elongate medical device to be inserted within a patient body, that according to some embodiments, includes an optical fiber extending between an optical interface at a proximal end of the medical device and a distal end of the medical device, where the optical fiber includes a single core fiber extending along a longitudinal length of the optical fiber. In such embodiments, the single core fiber is disposed radially offset from a central axis of the optical fiber, and the single core fiber is configured to receive an incident light via the optical interface and a propagate the incident light distally along the longitudinal length. The single core fiber includes a plurality of sensors distributed along the longitudinal length, where each sensor of the plurality of sensors is configured to (i) reflect a light signal of a different spectral width based on the incident light, and (ii) change a characteristic of the reflected light signal based on a state of the single core fiber, and where the state of the single core fiber is defined by a condition of the patient body.

Also disclosed herein is a method performed by a medical system, that according to some embodiments, includes projecting a light distally along an optical fiber of the system, the optical fiber disposed within a patient body, where the optical fiber includes a single core fiber disposed radially offset from a central axis of the optical fiber. The single core fiber is configured to (i) receive the light via an optical interface of the optical fiber coupled with a console of system and (ii) propagate the light distally along a longitudinal length of the single core fiber. The method further includes (i) receiving a light signal from the optical fiber, the light signal propagating proximally along the single core fiber, (ii) extracting from the light signal a number of conditions experienced by the single core fiber; and (iii) communicating the number of conditions to a user.

In some embodiments of the method, the number of conditions experienced by the single core fiber includes a damage to the single core fiber.

In some embodiments of the method, the number of conditions experienced by the single core fiber includes conditions of the patient body that include one or more of a flow velocity of a blood, a flow direction of the blood, a pH of the blood, or an oxygen level of the blood.

In some embodiments of the method, the single core fiber includes a plurality of sensors distributed along the longitudinal length, each sensor of the plurality of sensors configured to (i) define a reflected light signal of a different spectral width based on the light, and (ii) change a characteristic of the reflected light signal based on the number of conditions experienced by the single core fiber, and the number of conditions experienced by the single core fiber include one or more of (i) a core temperature of the patient body, (ii) a shape of the single core fiber defined by a shape of a blood vessel, (iii) a fluctuating motion of the single core fiber consistent with placement of a distal end the optical fiber adjacent a cavoatrial junction, or (iv) a compressive force applied longitudinally to the optical fiber resulting from contact of the distal end of the optical fiber with an anatomical element of the patient body.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an illustrative embodiment of a medical system including an elongate medical device having an optical fiber, in accordance with some embodiments;

FIG. 2 is an exemplary embodiment of a structure of a section of the elongate medical device of FIG. 1, in accordance with some embodiments;

FIG. 3A illustrates an embodiment of the elongate medical device of FIG. 1, in accordance with some embodiments;

FIG. 3B is a cross sectional end view of the elongate medical device of FIG. 3A, in accordance with some embodiments;

FIGS. 4A-4B are flowcharts of the methods of operations conducted by the medical system of FIG. 1 to achieve optical three-dimensional shape sensing, in accordance with some embodiments;

FIG. 5 is perspective illustration another embodiment of an optical fiber having a single core fiber disposed offset from a central axis of the optical fiber, in accordance with some embodiments;

FIG. 6A illustrates an implementation of the optical fiber of FIG. 5 determining a pH of a blood, in accordance with some embodiments;

FIG. 6B illustrates an implementation of the optical fiber of FIG. 5 determining engagement of the optical fiber with the check valves of a vein, in accordance with some embodiments;

FIG. 6C illustrates an implementation of the optical fiber of FIG. 5 determining an insertion length of the optical fiber, in accordance with some embodiments;

FIGS. 6D-6E illustrate an implementation of the optical fiber of FIG. 5 determining parameters of infusate delivery, in accordance with some embodiments;

FIGS. 6F-6G illustrate an implementation of the optical fiber of FIG. 5 determining blood flow parameters within a blood vessel based a wavelength shift between a light projected onto and a light reflected off of particles within the blood, in accordance with some embodiments;

FIG. 6H illustrates an implementation of the optical fiber of FIG. 5 determining an oxygen level of blood, in accordance with some embodiments; and

FIG. 7 illustrates a flow chart of a method performed by system, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

The terms “proximal” and “distal” refer to opposite ends of a medical device, including an optical fiber disclosed herein. As used herein, the proximal portion of an optical fiber is the portion nearest a practitioner during use or least inserted within a patient, while the distal portion is the portion at the opposite end. For example, the proximal end of the optical fiber is defined as the end closest to the practitioner during utilization of the optical fiber. The distal end is the end opposite the proximal end, along the longitudinal direction of the optical fiber, e.g., the end furthest inserted into the patient.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit (ASIC), etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random-access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the logic may be stored in persistent storage.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

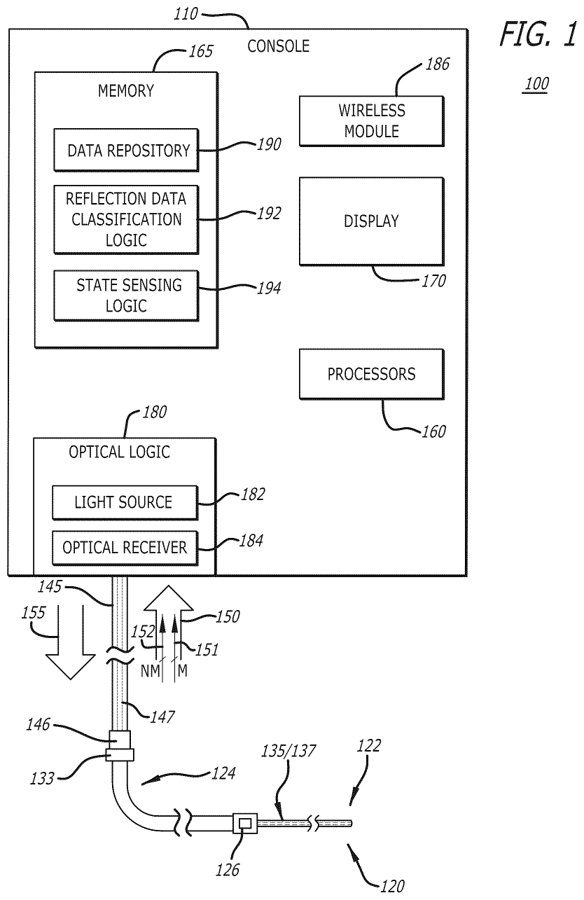

FIG. 1 illustrates an embodiment of a medical system including a medical device. As shown, the medical system (system) 100 generally includes a console 110 and an elongate medical device (device) 120 communicatively coupled with the console 110. The device 120 defines a distal end 122 and includes a console connector 133 at a proximal end 124. The device 120 includes an optical fiber 135 including multiple core fibers extending along a length of the device 120 as further described below. The console connector 133 enables the device 120 to be operably connected to the console 110 via an interconnect 145 including one or more optical fibers 147 (hereinafter, “optical fiber(s)”). Herein, the connector 146 is configured to engage (mate) with the console connector 133 to allow for the propagation of light between the console 110.

The device 120 may be configured to perform any of a variety of medical procedures. As such, the device 120 may be a component of or employed with a variety of medical instruments/devices 119. In some implementations, the device 120 may take the form of a guidewire, a stylet, or a catheter, for example. The device 120 may be formed of a metal, a plastic or a combination thereof. In some embodiments, the device 120 may include a lumen extending therealong having an optical fiber 135 disposed therein.

In some implementations, the device 120 may be integrated into a vascular catheter. Other exemplary implementations include drainage catheters, surgery devices, stent insertion and/or removal devices, biopsy devices, endoscopes, and kidney stone removal devices. In short, the device 120 may be employed with, or the device 120 may be a component of, any medical device 119 that is inserted into a patient.

According to one embodiment, the console 110 includes one or more processors 160, a memory 165, a display 170, and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The one or more processors 160, with access to the memory 165 (e.g., non-volatile memory or non-transitory, computer-readable medium), are included to control functionality of the console 110 during operation. As shown, the display 170 may be a liquid crystal diode (LCD) display integrated into the console 110 and employed as a user interface to display information to the clinician, especially during an instrument placement procedure. In another embodiment, the display 170 may be separate from the console 110. Although not shown, a user interface is configured to provide user control of the console 110.

Referring still to FIG. 1, the optical logic 180 is configured to support operability of the device 120 and enable the return of information to the console 110, which may be used to determine the physical state associated with the device 120 along or an image of the patient body. The physical state of the device 120 may be based on changes in characteristics of the reflected light signals 150 received at the console 110 from the device 120. The characteristics may include shifts in wavelength caused by strain on certain regions of the core fibers integrated within the optical fiber 135 positioned within or operating as the device 120, as shown below. As discussed herein, the optical fiber 135 may be comprised of a number (e.g., 1, 2, 3, 4, or more) of core fibers 137₁-137_{M} (M=1 for a single core, and M>2 for a multi-core), where the core fibers 137₁-137_{M} may collectively be referred to as core fiber(s) 137. Unless otherwise specified or the instant embodiment requires an alternative interpretation, embodiments discussed herein will refer to an optical fiber 135. From information associated with the reflected light signals 150, the console 110 may determine (through computation or extrapolation of the wavelength shifts) the physical state of the device 120 and/or physical conditions experienced by the probe 120, such as strain, temperature, pressure, or movement, for example.

According to one embodiment of the disclosure, as shown in FIG. 1, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the optical fiber 135 within the device 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the optical fiber 135 deployed within the device 120, and (ii) translate the reflected light signals 150 into reflection data (from a data repository 190), namely data in the form of electrical signals representative of the reflected light signals including wavelength shifts caused by strain. The reflected light signals 150 associated with different spectral widths may include reflected light signals 151 provided from sensors positioned in the center core fiber (reference) of the optical fiber 135 and/or reflected light signals 152 provided from sensors positioned in the periphery core fibers of the optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the one or more processors 160, which governs their operation. Also, the optical receiver 184 is operably coupled so as to provide the reflection data (from the data repository 190) to the memory 165 for storage and processing by reflection data classification logic 192. The reflection data classification logic 192 may be configured to: (i) identify which core fibers pertain to which of the received reflection data (from the data repository 190) and (ii) segregate the reflection data stored within the data repository 190 provided from reflected light signals 150 pertaining to similar regions of the device 120 or spectral widths into analysis groups. The reflection data for each analysis group is made available to state sensing logic 194 for analytics.

According to one embodiment of the disclosure, the state sensing logic 194 is configured to compare wavelength shifts measured by sensors deployed in each periphery core fiber at the same measurement region of the device 120 (or same spectral width) to the wavelength shift at a center core fiber of the optical fiber 135 positioned along central axis and operating as a neutral axis of bending. From these analytics, the state sensing logic 194 may determine the shape the core fibers have taken in three-dimensional space and may further determine the current physical state of the device 120 in three-dimensional space for rendering on the display 170.

According to one embodiment of the disclosure, the state sensing logic 194 may generate a rendering of the current physical state of the device 120, based on heuristics or run-time analytics. For example, the state sensing logic 194 may be configured in accordance with machine-learning techniques to access the data repository 190 with pre-stored data (e.g., images, etc.) pertaining to different regions of the device 120 in which reflected light from core fibers have previously experienced similar or identical wavelength shifts. From the pre-stored data, the current physical state of the device 120 may be rendered. Alternatively, as another example, the state sensing logic 194 may be configured to determine, during run-time, changes in the physical state of each region of the optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the optical fiber 135 to the wavelength shift generated by a sensor of the center core fiber at the same cross-sectional region. It is contemplated that other processes and procedures may be performed to utilize the wavelength shifts as measured by sensors along each of the core fibers within the optical fiber 135 to render appropriate changes in the physical state of the device 120, especially to enable guidance of the device 120 when positioned within the patient and at a desired destination within the body.

It is contemplated that other processes and procedures may be performed to utilize the wavelength shifts as measured by sensors along each of the core fibers within the optical fiber 135 to render appropriate changes in the physical state of the probe 120, especially to enable guidance of the probe 120 when positioned within the patient and at a desired destination within the body. For example, wavelength shifts as measured by sensors along one or more of the core fibers may be based on physical states or condition of the probe 120 other than or in addition to longitudinal strain experienced by the device 120. Alternative or additional physical states may include one or more of torsional strain, temperature, motion, oscillations, pressure, or fluid flow adjacent the elongate medical device.

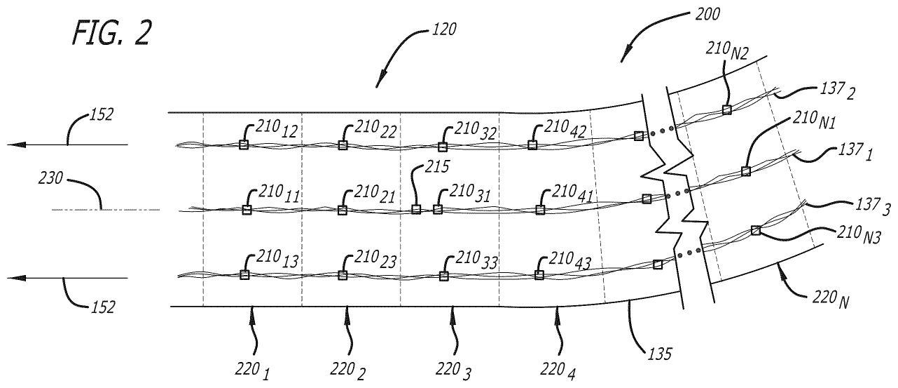

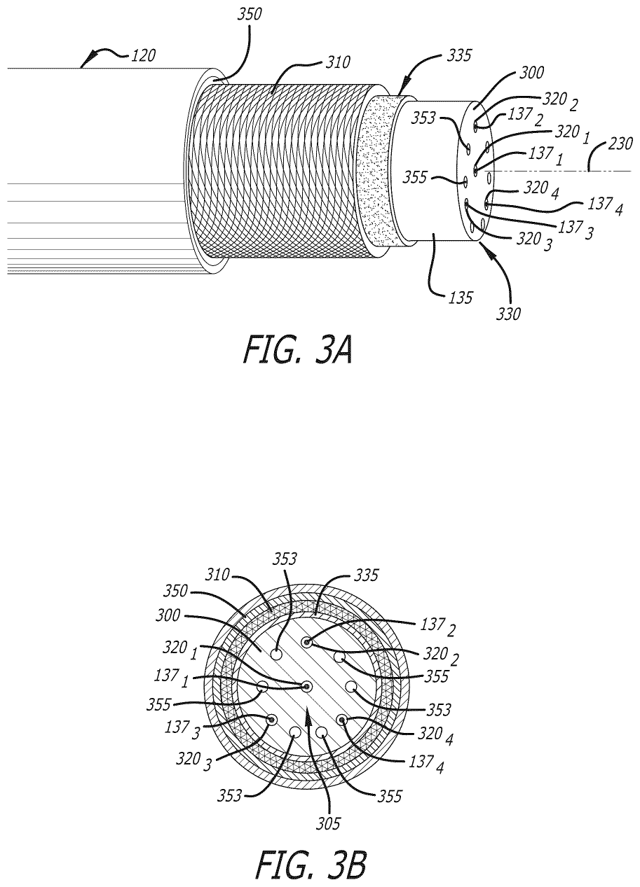

Referring to FIG. 2, an exemplary embodiment of a structure of a section of the optical fiber of FIG. 1 is shown in accordance with some embodiments. The optical fiber section 200 of the optical fiber 135 depicts certain core fibers 137₁-137_{M} (M>2, M=4 as shown, see FIG. 3A) along with the spatial relationship between sensors (e.g., reflective gratings) 210₁₁-210_{NM} (N>2; M>2) present within the core fibers 137₁-137_{M}, respectively. As noted above, the core fibers 137₁-137_{M} may be collectively referred to as “the core fibers 137.”

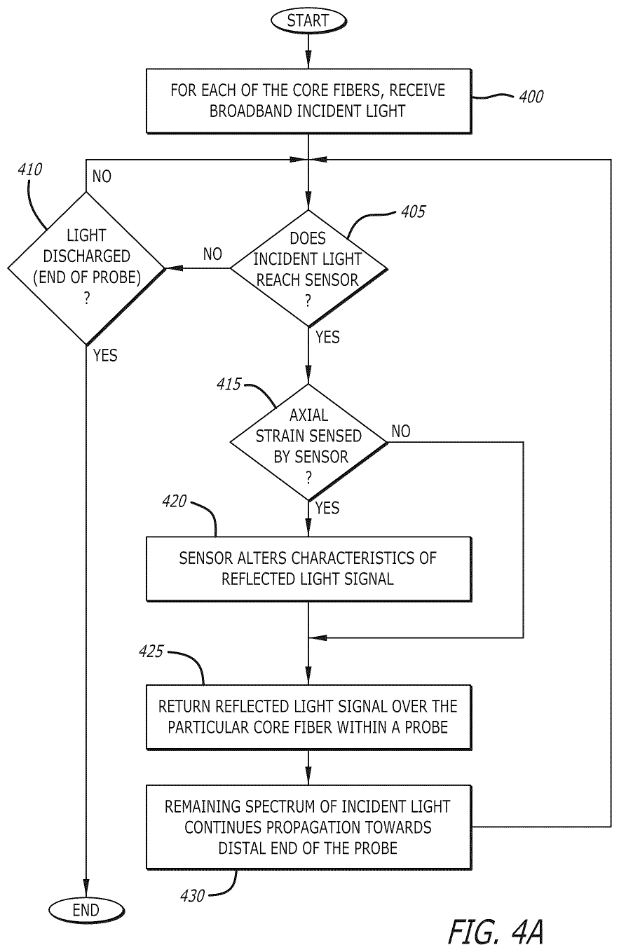

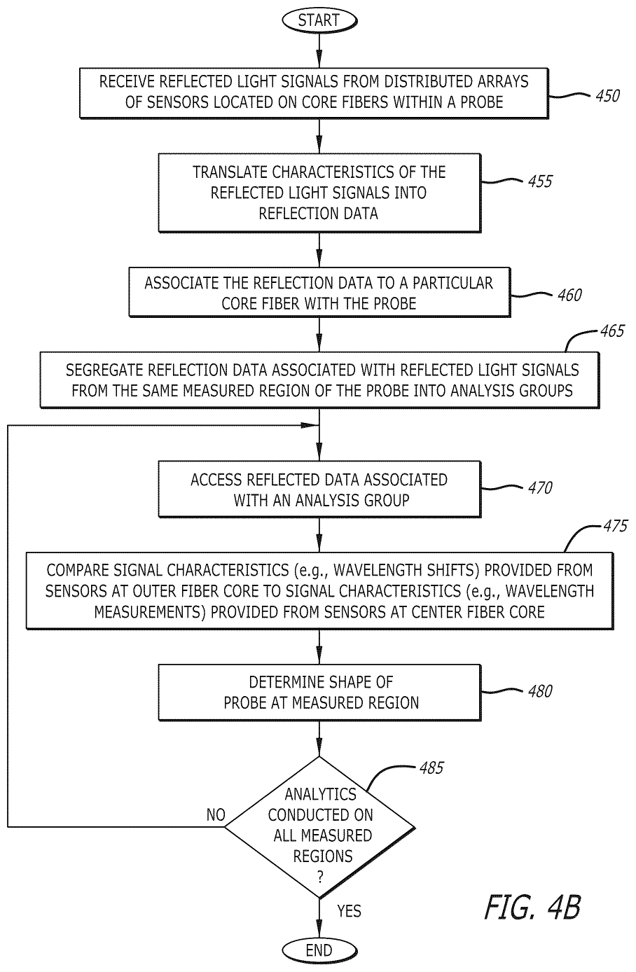

As shown, the section 200 is subdivided into a plurality of cross-sectional regions 220₁-220_{N}, where each cross-sectional region 220₁-220_{N} corresponds to reflective gratings 210₁₁-210₁₄ . . . 210_{N1}-210_{N4}. Some or all of the cross-sectional regions 220₁ . . . 220_{N} may be static (e.g., prescribed length) or may be dynamic (e.g., vary in size among the regions 220₁ . . . 220_{N}). A first core fiber 137₁ is positioned substantially along a center (neutral) axis 230 while core fiber 137₂ may be oriented within the cladding of the optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-4B provides illustrations of such.

Referencing the first core fiber 137₁ as an illustrative example, when the device 120 (see FIG. 1) is operative, each of the reflective gratings 210₁-210_{N} reflects light for a different spectral width. As shown, each of the gratings 210₁ᵢ-210_{Ni} (1<i<M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the optical fiber 135, the gratings 210₁₂-210_{N2} and 210₁₃-210_{N3} are configured to reflect incoming light at the same (or substantially similar) center frequency. As a result, the reflected light returns information that allows for a determination of the physical state of the core fibers 137 (and the device 120) based on wavelength shifts measured from the returned, reflected light about the center frequency. In particular, strain (e.g., compression or tension) applied to the optical fiber 135 (e.g., at least core fibers 137₂-137₃) results in wavelength shifts associated with the returned, reflected light. Based on different locations, the core fibers 137₁-137₄ experience different types and/or degrees of strain based on angular path changes as the device 120 advances in the patient.

For example, with respect to the multi-core optical fiber section 200 of FIG. 2, in response to angular (e.g., radial) movement of the device 120 is in the left-veering direction, the fourth core fiber 137₄ (see FIG. 3A) of the optical fiber 135 with the shortest radius during movement (e.g., core fiber closest to a direction of angular change) would exhibit compression (e.g., forces to shorten length). At the same time, the third core fiber 137₃ with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fiber 137₂ and 137₃ will exhibit different changes in wavelength. The differences in wavelength shift of the reflected light signals 150 can be used to extrapolate the physical configuration of the device 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the optical fiber 135. These degrees of wavelength change may be used to extrapolate the physical state of the device 120. The reflected light signals 150 are reflected back to the console 110 via individual paths over a particular core fiber 137₁-137_{M}.

Referring to FIG. 3A, a first exemplary embodiment of the probe of FIG. 1 supporting both an optical and electrical signaling is shown in accordance with some embodiments. Herein, the probe 120 features a centrally located multi-core optical fiber 135, which includes a cladding 300 and a plurality of core fibers 137₁-137_{M} (M>2; M=4) residing within a corresponding plurality of lumens 320₁-320_{M}. While the multi-core optical fiber 135 is illustrated within four (4) core fibers 137₁-137₄, a greater number of core fibers 137₁-137_{M}(M>4) may be deployed to provide a more detailed three-dimensional sensing of the physical state (e.g., shape, etc.) of the multi-core optical fiber 135 and the probe 120 deploying the optical fiber 135.

In some embodiments, the multi-core optical fiber 135 is encapsulated within a concentric braided tubing 310 positioned over a low coefficient of friction layer 335. The braided tubing 310 may feature a “mesh” construction, in which the spacing between the intersecting conductive elements is selected based on the degree of rigidity desired for the probe 120, as a greater spacing may provide a lesser rigidity, and thereby, a more pliable probe 120.

According to this embodiment of the disclosure, as shown in FIGS. 3A-3B, the core fibers 137₁-137₄ include (i) a central core fiber 137₁ and (ii) a plurality of periphery core fibers 137₁-137₄, which are maintained within lumens 320₁-320₄ formed in the cladding 300. According to one embodiment of the disclosure, one or more of the lumen 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 320₁-320₄, the wavelength changes to the incident light are caused by angular deviations in the multi-core optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₁-320₄ each formed within different areas of the cladding 300 radiating from the first neutral axis 230. In general, the core fibers 137₁-137₄, exclusive of the central core fiber 137₁, may be positioned at different areas within a cross-sectional area 305 of the cladding 300 to provide sufficient separation to enable three-dimensional sensing of the multi-core optical fiber 135 based on changes in wavelength of incident light propagating through the core fibers 137₁-137₄ and reflected back to the console for analysis.

For example, where the cladding 300 features a circular cross-sectional area 305 as shown in FIG. 3B, the core fibers 137₁-137₄ may be positioned substantially equidistant from each other as measured along a perimeter of the cladding 300, such as at “top” (12 o'clock), “bottom-left” (8 o'clock) and “bottom-right” (4 o'clock) locations as shown. Hence, in general terms, the core fibers 137₁-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, operating as the conductive medium for the probe 120, the braided tubing 310 may provide mechanical integrity to the multi-core optical fiber 135 and may also operate as a conductive pathway for electrical signals. The cladding 300 and the braided tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both for the cladding 300 and the braided tubing 310, as shown.

Referring to FIGS. 4A-4B, flowcharts of methods of operations conducted by the medical device system of FIG. 1 to achieve optic three-dimensional shape sensing are shown in accordance with some embodiments. The first micro-lumen is coaxial with the central axis of the probe. The first micro-lumen is configured to retain a center core fiber. Two or more micro-lumen, other than the first micro-lumen, are positioned at different locations circumferentially spaced along the circumferential edge of the probe. For example, two or more of the second plurality of micro-lumens may be positioned at different quadrants along the circumference edge of the probe.

Furthermore, each core fiber includes a plurality of sensors spatially distributed along its length between at least the proximal and distal ends of the probe. This array of sensors is distributed to position sensors at different regions of the core fiber to enable distributed measurements of strain throughout the entire length or a selected portion of the probe. These distributed measurements may be conveyed through reflected light of different spectral widths (e.g., specific wavelength or specific wavelength ranges) that undergoes certain wavelength shifts based on the type and degree of strain, including oscillations of the strain.

According to one embodiment of the disclosure, as shown in FIG. 4A, for each core fiber, broadband incident light is supplied to propagate through a particular core fiber (block 400). Unless discharged, upon the incident light reaching a sensor of a distributed array of sensors measuring strain on a particular core fiber, light of a prescribed spectral width associated with the first sensor is to be reflected back to an optical receiver within a console (blocks 405-410). Herein, the sensor alters characteristics of the reflected light signal to identify the type and degree of strain on the particular core fiber as measured by the first sensor (blocks 415-420). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the probe (blocks 425-430). The remaining spectrum of the incident light may encounter other sensors of the distributed array of sensors, where each of these sensors would operate as set forth in blocks 405-430 until the last sensor of the distributed array of sensors returns the reflected light signal associated with its assigned spectral width and the remaining spectrum is discharged as illumination.

Referring now to FIG. 4B, during operation, multiple reflected light signals are returned to the console from each of the plurality of core fibers residing within the corresponding plurality of micro-lumens formed within a probe. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 450-455). The reflection data classification logic is configured to identify which core fibers pertain to which reflection data and segregate reflection data provided from reflected light signals pertaining to a particular measurement region (or similar spectral width) into analysis groups (block 460-465).

Each analysis group of reflection data is provided to sensing logic for analytics (block 470). Herein, the sensing logic compares wavelength shifts at each outer core fiber with the wavelength shift at the center core fiber positioned along central axis and operating as a neutral axis of bending (block 475). From this analytics, on all analytic groups (e.g., reflected light signals from sensors in all or most of the core fibers), the sensing logic may determine the shape the core fibers have taken in three-dimensional space, from which the sensing logic can determine the current physical state of the probe in three-dimensional space (blocks 480-485).

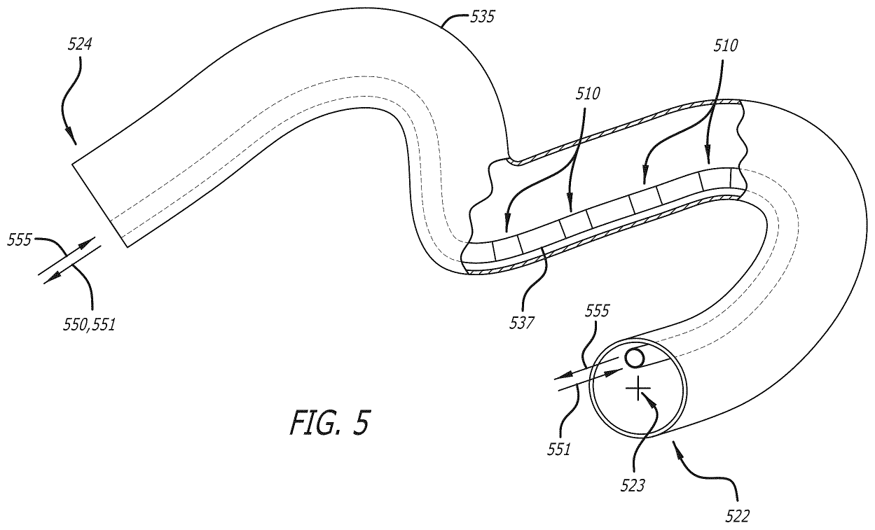

Referring to FIG. 5, illustrates a second exemplary embodiment of an optical fiber 535 that can, in certain respects, resemble components of the optical fiber 135 described in connection with FIGS. 1-3B. It will be appreciated that all the illustrated embodiments may have analogous features. Accordingly, like features are designated with like reference numerals, with the leading digits of “5.” For instance, the core fibers are designated as “137” in FIGS. 1-3B, and analogous core fibers are designated as “537” in FIG. 5. Relevant disclosure set forth above regarding similarly identified features thus may not be repeated hereafter. Moreover, specific features of the optical fiber 135 and related components shown in FIGS. 1-3B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the optical fiber 535 of FIG. 5. Any suitable combination of the features, and variations of the same, described with respect to the optical fiber 135 and components illustrated in FIGS. 1-3B can be employed with the optical fiber and components of FIG. 5, and vice versa.

The optical fiber 535 includes a single core fiber 537 having a number (e.g., at least 1, 5, 10, 20, 40 or more) of sensors (e.g., reflective gratings) 510 disposed along a length of the optical fiber 535. The single core fiber 537 is disposed radially offset from a central axis 523 of the optical fiber 535. The optical fiber 535 is configured for insertion within the vasculature of the patient, such as along with or as part of the device 120. In some embodiments, the optical fiber 535 may disposed within a lumen of a catheter and in some embodiments, the optical fiber 535 may be embedded within or extend along a wall of the catheter.

The optical fiber 535 is generally configured to determine a shape or movement (i.e., changing shape) of the optical fiber 535 or portion thereof which by association may include the device 120 or portion thereof. The single core fiber 537 is disposed radially offset from a central axis 523 of the optical fiber 535. As such, bending (or otherwise changing a curvature of) the optical fiber 535 may generate a strain of the core fiber 537, where the strain is detectable via the sensors 510 disposed along a length of the core fiber 537.

The core fiber 537 may also be configured to (i) project an excitation light 551 distally away from the distal end 522 of the optical fiber 535 and (ii) receive an excitation light signal 555 via the distal end 522. In some embodiments, the excitation light 551 may be an illuminating light and the excitation light signal 555 may include an image of an anatomical element of the patient body or other object adjacent the distal end 522.

The sensors 510 or a subset thereof may be configured to determine longitudinally compressive strain along a portion of the optical fiber 535. For example, a subset of sensors adjacent the distal tip 522 may be configured to detect (i) engagement/contact with check valves of a vein or (ii) abutment with other anatomical elements, such as a blood vessel wall, for example.

In some embodiments, the orientation (i.e., angular position) of the core fiber 537 may be known. For example, the device 120 may include a feature (e.g., an indicium) indicating the angular position of the core fiber 537. For example, the device 120 may be configured so that, when the indicum is vertically oriented, the core fiber 537 is oriented at the 12 o-clock position. As such, during use, the clinician may rotate device 120 about its longitudinal axis and thereby, correspondingly orient the single core fiber 537 in a desired direction.

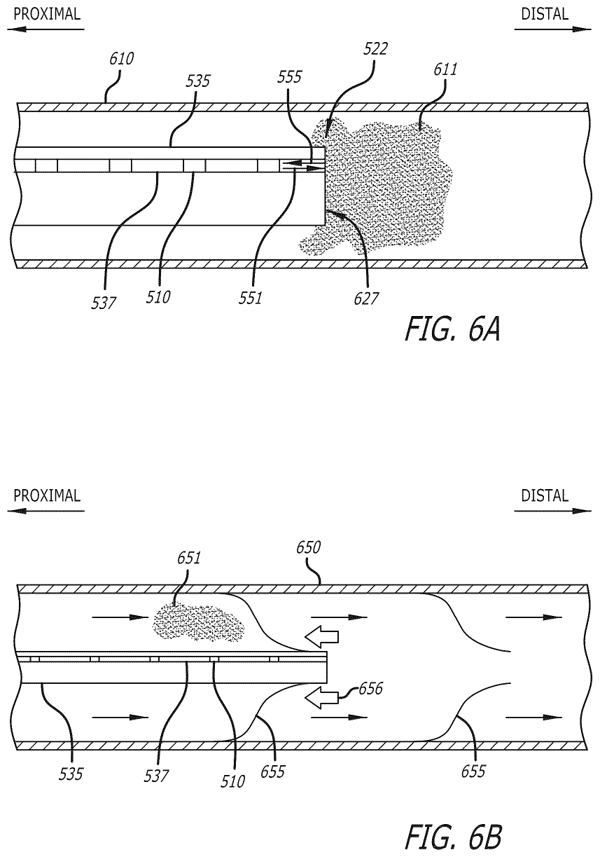

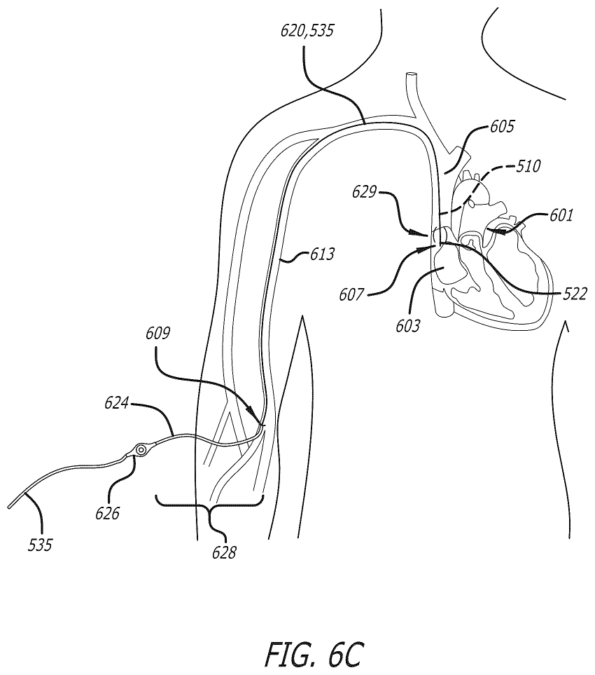

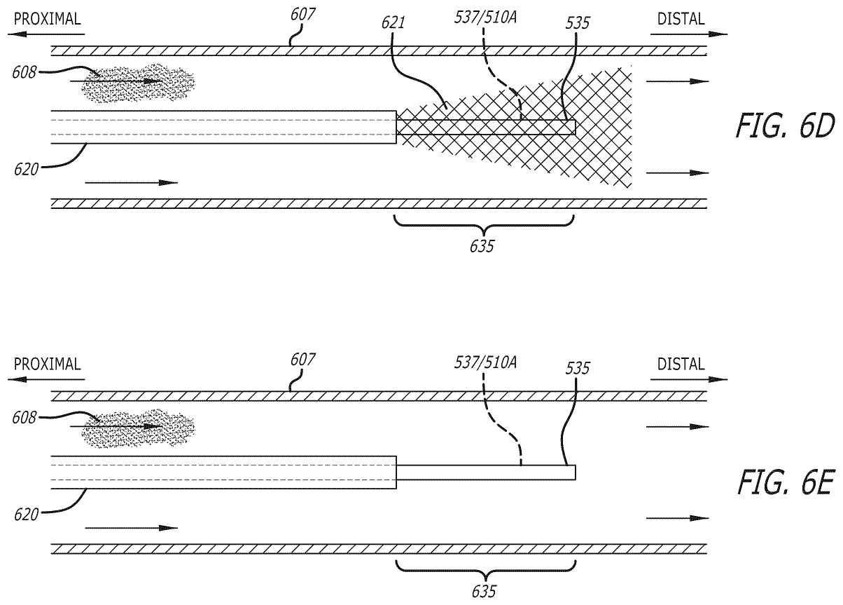

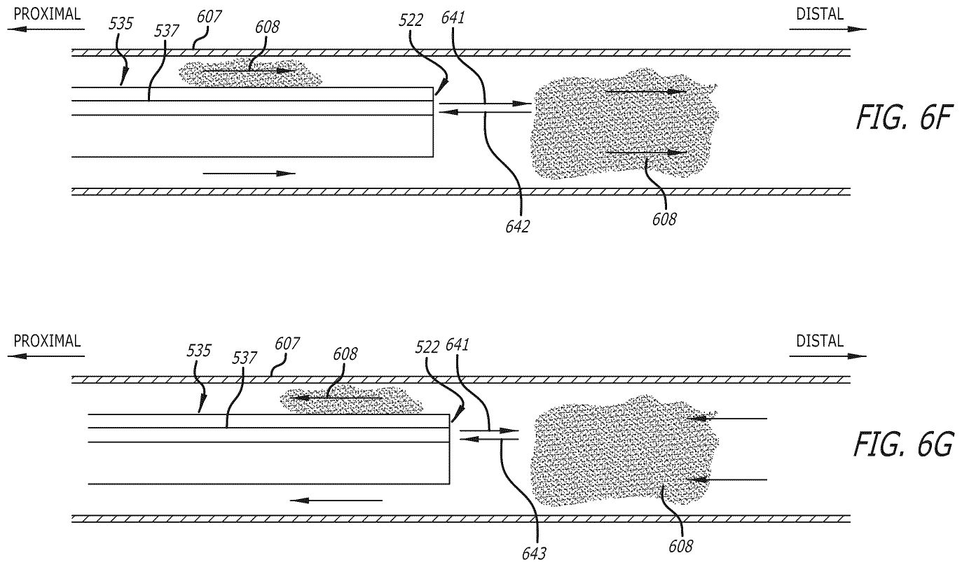

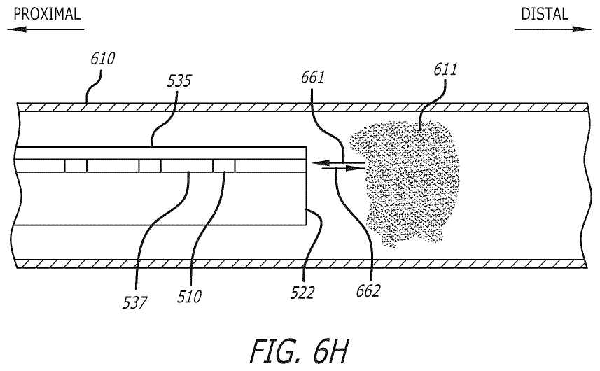

FIG. 6A illustrates a first implementation of the optical fiber 535 in combination with the state sensing logic 194 determining a pH of a solution. FIG. 6A shows the optical fiber 535 disposed within a blood vessel 610 such that the distal end 522 is in contact with the blood 611. In some embodiments, the optical fiber 535 may include a coating 627 disposed across a distal face of the optical fiber 535. The coating 627 may be configured to define a characteristic of the excitation light signal 555 based on a pH of a solution (e.g., the blood 611) in contact with the coating 627. In some embodiments, the characteristic of the excitation light signal 555 may be a color. By way of one example, the coating 627 may be configured to function similar to a litmus paper that changes color in accordance with a pH of a solution in contact with the litmus paper. In some embodiments, the coating 627 may be applied to a surface of the device 120 other than the optical fiber 535 directly.

In some embodiments, the system may determine a pH of a solution in contact with the coating 627. In some embodiments of use, the solution may be the blood 611, while in other embodiments, the solution may be some other solution, such as an infusate, or urine, for example. In some embodiments, the state logic 194 may project the excitation light 555 distally along the single core fiber 537 so as to optically excite (e.g., illuminate) the coating 627. The state logic 194 may receive the excitation light signal 555 including a characteristic of the excitation light signal 555 that is based on the pH. The state logic 194 may further extract pH data from the excitation light signal 555 and communicate the pH to the user, such as via the display 170, for example.

FIG. 6B illustrates a second implementation of the optical fiber 535 in combination with the state sensing logic 194 identifying check valves along a blood vessel, such as are present along a vein. FIG. 6B shows a vein 650 having venous blood 651 flowing through check valves 655. The optical fiber 535 is shown inserted into the vein 650 in the direction of flow of the venous blood 651. The optical fiber 535 is further shown inserted through one of the check valves 655.

During advancement of the optical fiber 535 along the vein 650, the optical fiber 535 may engage/contact the check valve 655 such that the check valve 655 exerts a force 656 onto the optical fiber 535. The optical fiber 535 experiences a longitudinally directed compressive strain as a result of the exerted force 656. The sensors 510, in turn, may define the varying reflected light signals based on the compressive strain of the optical fiber 535 induced by the exerted force 656.

The state sensing logic 194 may receive electrical signals related the reflected light signals during the insertion process of the optical fiber 535. As the optical fiber 535 engages the check valve 655 during insertion, the electrical signals may indicate a change in the reflected light signals based on the compressive strain resulting from engagement of the optical fiber 535 with the check valve 655 and define present engagement data therefrom. For example, during insertion the state sensing logic 194 may determine a first level of compressive strain as the optical fiber is advanced between adjacent check valves 655 and then determine an increased second level (e.g., a spike) of compressive strain as the optical fiber 535 engages/contacts each check valve 655. In some embodiments, the state sensing logic 194 compare a present difference in magnitude between the second level of compressive strain and the first level of compressive strain with a difference in magnitude stored in memory. As a result of the comparison, the state sensing logic 194 may identify check valves 655 along a blood vessel.

According to a similar implementation, in some instances during insertion of the optical fiber 535, the distal end 522 may abut other anatomical elements, such as a blood vessel wall, for example. In some instances, continued insertion may cause harm to the patient. As such, the state sensing logic 194 may identify the abutment and alert the user to prevent the harm. The state sensing logic 194 may receive electrical signals related the reflected light signals during the insertion process of the optical fiber 535. When the optical fiber 535 abuts an anatomical element during insertion, the electrical signals may indicate a change in the reflected light signals based on the compressive strain resulting from the abutment and define present abutment data therefrom. For example, during insertion the state sensing logic 194 may determine an increased level (e.g., a spike) of compressive strain when the optical fiber 535 abuts the anatomical element. In some embodiments, the state sensing logic 194 compare the present increased level of compressive strain with a safety limit stored in memory. As a result of the comparison, the state sensing logic 194 may identify the abutment and provide an alert to the user.

FIG. 6C illustrates a third implementation of the optical fiber 535 in combination with the state sensing logic 194 determining the location of a cavoatrial junction (CAJ) of the patient with respect to an insertion site of a catheter, such as a peripherally inserted central catheter (PICC), for example. In other words, the state sensing logic 194 in combination with the optical fiber 535 may determine a length of a vascular pathway from the insertion site to the CAJ. FIG. 6C shows a vascular pathway 613 extending between an insertion site 609 and a heart 601. Also shown is the CAJ 607 disposed between the superior vena cava 605 and the right atrium 603. A PICC 624 having a hub 626 is inserted into the vascular pathway 613. An excess length 628 of the PICC 624 is also shown extending between the insertion site 609 and the hub 626. The optical fiber 535 is also inserted with the vascular pathway 613 so that the distal end 522 is disposed adjacent the CAJ 607.

In some instances, it may be beneficial to shorten/trim the PICC 624 so as to minimize the excess length 628. In accordance with one exemplary implementation, the optical fiber 535 which may be incorporated into a guidewire may be inserted into the vascular pathway 613 until a fluctuating motion of the optical fiber is detected by the sensors 510 of the optical fiber 535 located adjacent the distal end 622. The fluctuating motion may be caused by oscillating motion of anatomical elements adjacent the CAJ 607, such as fluctuating blood pressure or flow, or oscillating motion of heart tissue or tissue adjacent the heart, for example. In other words, the state sensing logic 194 may be configured to provide notification to the user when the optical fiber 535 is sufficiently inserted to locate the distal end 522 adjacent the CAJ 607.

The state sensing logic 194 may then determine an insertion length of the optical fiber 535 disposed within the vascular pathway 613. According to one exemplary implementation, the state sensing logic 194 may determine the insertion length via the blood temperature sensed along the insertion length versus to the room temperature sensed along an excess length of the optical fiber 535 disposed outside of the vascular pathway 613. In some embodiments, the state sensing logic 194 may communicate the insertion length to the user via the display 170. The user may then trim the PICC in accordance with the determined insertion length. The user may also thread the PICC 624 along the optical fiber 535 to advance the PICC 624 to the CAJ 607. As may be appreciated by one or ordinary skill, the state sensing logic 194 may utilize any other sensing capabilities of the optical 535 described above to locate the distal end 522 adjacent the CAJ 607 and/or determine the insertion length.

FIGS. 6D-6E illustrate a fourth implementation of the optical fiber 535 in combination with the state sensing logic 194 determining parameters of an infusate delivery. As an infusate is delivered to a blood vessel via a catheter, the infusate is generally mixed with the blood downstream of the point of infusion (e.g., the distal end of the catheter). As the infusate may have a temperature different that the blood, the temperature of the blood portion having infusate mixed therewith (i.e., the blood portion downstream of the infusion point) may be measurably different than the temperature of the blood portion having no infusate mixed therewith (i.e., the blood portion upstream of the infusion point). As such, delivery parameters of the infusate may be determined, such as the starting and stopping of delivery.

In the illustrated embodiment, at least a subset of the sensors 510 of the optical fiber 535 are configured to detect temperature, e.g., determine a temperature of a substance, such as blood 608, for example, adjacent the optical fiber 535 at the locations of the respective sensors 510. In some embodiments, determining a temperature may include the sensors 510 detecting a temperature induced strain of the optical fiber 535 due to thermal expansion/contraction.

FIG. 6D illustrates a catheter 620 delivering an infusate 621 to a blood vessel 607 and FIG. 6E illustrates the catheter 620 not delivering an infusate 621 to the blood vessel 607. The optical fiber 535 extends along the catheter 620 (e.g., inserted within a lumen of the catheter 620) such that a section 635 of the optical fiber 535 along with a subset 510A of sensors 510 extend beyond the catheter 620. Each sensor 510 of the subset 510A are configured to determine temperature. As such, the subset 510A of sensors 510 sense a temperature of a mixture of infusate 621 and blood 608 during delivery of the infusate (FIG. 6C). Similarly, the subset 510A of sensors 510 sense a temperature of the blood 608 only during non-delivery of the infusate (FIG. 6D).

In some embodiments, the state sensing logic 194 may receive a first reflected light signal from the subset 510A during non-delivery of the infusate 621, the first reflected light signal based on the first present temperature of the blood 608. The state sensing logic 194 may receive a second reflected light signal from subset 510A during delivery of the infusate 621, the second reflected light signal based on a present second temperature of the blood 608 mixed with the infusate 621. In some embodiments, the state sensing logic 194 may compare the first and second present temperatures with a temperature limit stored in the non-transitory computer-readable storge medium and as a result of the comparison determine when the infusate 621 is delivered versus non-delivered.

Similarly, the state sensing logic 194 may determine when delivery is initiated and terminated, where initiating and terminating the delivery of the infusate defines a flushing event. In such embodiments, the state sensing logic 194 may determine a number and or frequency of flushing events. In a similar fashion, as the delivery rate of infusate may be known, the state sensing logic 194 may determine a delivery volume of infusate. In an alternative implementation, the delivery of the infusate may include individually infused known volumes (e.g., volumes delivered by multiple syringes). In such an implementation, the state sensing logic 194 may count the number of individually infused known volumes and determine therefrom a total infused volume of infusate.

FIGS. 6F-6G illustrate a fifth implementation of the optical fiber 535 in combination with the state sensing logic 194 determining blood flow parameters based on reflections of incident light that are shifted toward the red or blue spectrum. The optical fiber 535 may be advanced along a blood vessel in the same direction as the blood flow or in the opposite direction to the blood flow. In accordance with the doppler effect, reflections of incident light projected onto particles within the blood may shift toward the red spectrum (i.e., longer wavelengths) when the blood is flowing away from the light source. Conversely, reflections of incident light projected onto particles within the blood may shift toward the blue spectrum (i.e., shorter wavelengths) when the blood is flowing toward from the light source. As such, an analysis of the reflected light may indicate a direction of blood flow. In a similar fashion (i.e., via the doppler effect), the state sensing logic 194 may also determine a velocity of the blood 608 flow in either the distal direction or proximal direction with respect to the optical fiber 535.

FIGS. 6F and 6G illustrate the optical fiber 535 inserted within the blood vessel 607 such that a distal end 522 of the optical fiber 535 is disposed within the blood 608 flowing within the blood vessel 607. An incident light 641 is propagated distally along the optical fiber 535 and projected distally away from the distal end 522 into the blood 608, i.e., onto particles within the blood 608. The incident light 641 reflects off of the particles within the blood 608 to generate reflected light that is received by the optical fiber 535 and propagated proximally back along the optical fiber 535.

FIG. 6F illustrates a first instance, where the blood 608 is flowing in a distal direction with respect to the optical fiber 535. As such, the incident light 641 is projected onto blood particles that are moving away from the distal end 522 generating reflected light 642 that has shifted toward the red spectrum with respect to the incident light 641.

FIG. 6G illustrates a second instance, where the blood 608 is flowing in a proximal direction with respect to the optical fiber 535. As such, the incident light 641 is projected onto blood particles that are moving toward the distal end 522 generating reflected light 643 that has shifted toward the blue spectrum with respect to the incident light 641.

The state sensing logic 194 may receive electrical signals related to the reflected light propagated proximally along the optical fiber 535, where the electrical signals indicate a wavelength of the reflected light. In some embodiments, the state sensing logic 194 may compare the present wavelength of the reflected light with a wavelength of the incident light 641. As a result of the comparison, the state sensing logic 194 may determine (i) that the blood 608 is flowing in a distal direction with respect to the optical fiber 535 or (ii) that the blood 608 is flowing in a proximal direction with respect to the optical fiber 535. In some embodiments, the state sensing logic 194 may compare the present wavelength of the reflected light with a wavelength of the incident light 641 and determine therefrom a blood flow velocity.

FIG. 6H illustrates a sixth implementation of the optical fiber 535 in combination with the state sensing logic 194 determining an oxygen level of the blood. FIG. 6H shows the optical fiber 535 disposed within the blood vessel 610 such that the distal end 522 is in contact with the blood 611. An excitation light 661 is projected away from the distal end 522 of the optical fiber 535 into the blood 611. The excitation light 661 may comprise wavelengths consistent with generating a return light signal 662 consistent with an oxygen level (oxygen saturation) of the blood 611. The state sensing logic 194 may extract the oxygen level of the blood 611 from the return light signal 662. The state sensing logic 194 may communicate the oxygen level of the blood 611 to the user, such as via the display 170, for example.

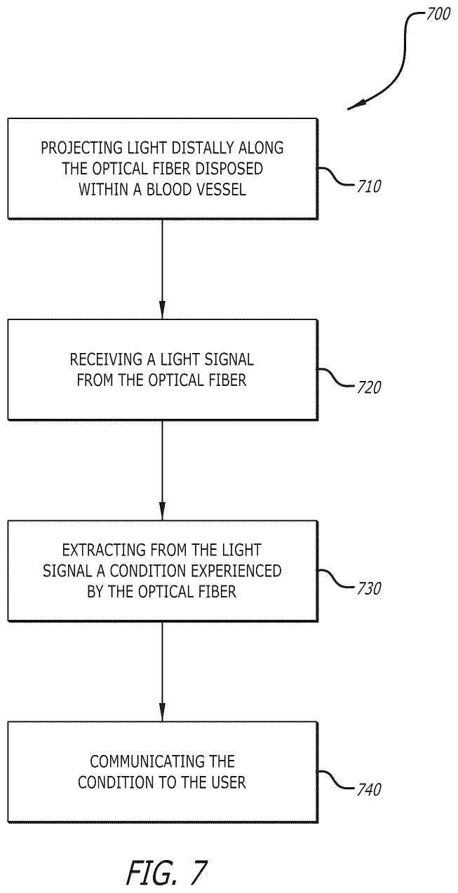

FIG. 7 illustrates a flow chart of a method 700 performed by the medical system 100 that may, according to some embodiments, include all or a subset of the flowing steps or process. The method 700 includes projecting a light distally along an optical fiber of the system (block 710). Projecting the light may include projecting the light away from a distal end of the optical fiber disposed within a blood vessel. In some embodiments, the projected light may include broadband light or specific wavelengths.

The method 700 further includes receiving light signals from the optical fiber (block 720). Receiving the light signal may include receiving the light signal from into the optical fiber via the distal end of the optical fiber. Receiving the light signal may also include receiving reflected light signals defined by sensors disposed along the optical fiber in response to the projected light. In some embodiments, receiving the light signal my include receiving multiple light signals.

The method 700 further includes extracting from the light signal a number of conditions experienced by the optical fiber (block 730). In some embodiments, the number of conditions experienced by the optical fiber may include a damage to the optical fiber. The conditions may also include a compressive force applied longitudinally to the optical fiber resulting from contact of the distal end of the optical fiber with an anatomical element of the patient body. The conditions may also include a shape of the optical fiber which may be defined by a shape of a blood vessel, for example.

In some embodiments, the number of conditions experienced by the optical fiber includes conditions of the patient body that include one or more of a velocity and or direction of blood flow with respect to the optical fiber. The conditions of the patient body may also include a pH of a fluid within the body, such as blood, urine, infusate, or any other bodily fluid. The conditions of the patient body may also include an oxygen level of the blood. The conditions of the patient body may also include a fluctuating motion of the optical fiber as caused by fluctuating blood flow or pressure within a vasculature. The conditions of the patient body may also include a core temperature of the patient.

The method 700 further includes communicating the extracted conditions to a user (block 740). The communication may include information rendered on the display, such as data, an alert, an image, or the like.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical system, comprising:
an elongate medical device including an optical fiber having a single core fiber extending along the optical fiber, the single core fiber disposed radially offset from a central axis of the optical fiber, the single core fiber including a plurality of sensors distributed along a longitudinal length of the single core fiber, each sensor of the plurality of sensors configured to reflect a light signal of a different spectral width based on received incident light, and change a characteristic of the reflected light signal based on a state of the optical fiber; and
a console including:
a light source optically coupled with the optical fiber via an optical connector;
an optical receiver optically coupled with the optical fiber via the optical connector;
a display;
a processor; and
a non-transitory computer-readable medium having stored thereon logic that, when executed by the processor, causes operations including:
projecting via the light source a light distally along the optical fiber;
receiving via the optical receiver the reflected light signal from the plurality of sensors;
processing the reflected light signal by the processor to determine the state of the optical fiber based on the reflected light signal; and
communicating the state to a user via the display.

2. The system according to claim 1, wherein:
the state of the optical fiber includes a compressive strain of the optical fiber caused by a contact force applied to the distal end during advancement of the medical device along the blood vessel, and
the operations further include:
extracting from the at least one reflected light signal present compressive strain data; and
identifying from the compressive strain data check valves disposed along the blood vessel.

3. The system according to claim 1, wherein:
the state of the optical fiber includes a compressive strain of the optical fiber caused by a contact force applied to the distal end of the optical fiber, and
the operations further include:
extracting from the at least one reflected light signal present compressive strain data;
comparing the present compressive strain data with a compressive strain safety limit stored in the non-transitory computer-readable medium, the compressive strain safety limit defining a safe limit for contact of the distal end with the anatomical elements; and
providing an alert to the user when the present compressive strain data exceeds the compressive strain safety limit.

4. The system according to claim 1, wherein:
the state of the optical fiber includes a temperature experienced by one or more of the plurality of sensors when the optical fiber is inserted within the patient; and
the operations further include:
receiving one or more reflected light signals from the one or more sensors;
extracting present temperature data from the one or more reflected light signals; and
determining a core temperature of the patient from the present temperature data.

5. The system according to claim 1, wherein:
the optical fiber is inserted within the blood vessel,
the optical fiber extends along a catheter, the catheter configured to deliver an infusate to the blood vessel,
the state of the optical fiber includes:
a first temperature experienced by a section of the optical fiber extending beyond a distal end of the catheter during non-delivery of the infusate; and
a second temperature experienced by the section during delivery of the infusate; and
the operations further include:
receiving a first reflected light signal from a sensor disposed along the section during non-delivery of the infusate, the first reflected light signal based on the first temperature;
receiving a second reflected light signal from the sensor, the second reflected light signal based on the second temperature;
extracting from the first and second reflected light signals present temperature difference data between the first and second temperatures;
comparing the present temperature difference data with a temperature difference limit stored in the non-transitory computer-readable medium; and
determining, as result of the comparison, whether the infusate is delivered.

6. The system according to claim 5, wherein:
the infusate is a flushing solution for the catheter,
initiating and terminating the delivery of the infusate defines a flushing event; and
the operations further include determining at least one of a frequency or a number of flushing events.

7. The system according to claim 5, wherein:
the delivery rate of the infusate is known;
initiating and terminating the delivery of the infusate defines an infused volume of the infusate; and
the operations further include determining of the infused volume.

8. The system according to claim 1, wherein:
the state of the optical fiber includes:
a damaged optical fiber; and
the operations further include:
determining a damage of the optical fiber, wherein determining the damage includes at least one of:
receiving a subset of reflected light signals from a respective subset of sensors, the subset of reflected light signals having a spectral width that exceeds a defined spectral-width range stored in the non-transitory computer-readable medium, or
not receiving a reflected light signal from at least one sensor.

9. The system according to claim 8, wherein the operations further include:
determining a longitudinal location of the respective subset of sensors or the at least one sensor along the along the optical fiber, and
relating the longitudinal location of the respective subset of sensors or the at least one sensor with a longitudinal location of the damage along the optical fiber.

10. The system according to claim 1, wherein:
the optical fiber is inserted within the blood vessel, and
the operations further include:
projecting a light defining a first wavelength distally away from a distal end of the optical fiber into blood of the blood vessel;
receiving a reflected light signal having a second wavelength via the distal end;
extracting from the reflected light signal a present wavelength shift between the first wavelength and the second wavelength;
comparing the present wavelength shift with one or more wavelength shift limits stored in the non-transitory computer-readable medium; and
determining, as result of the comparison, at least one of a direction of blood flow or a velocity of the blood flow.

11. The system according to claim 1, wherein the operations further include:
projecting the light toward a coating of the medical device, the coating configured to define a light characteristic based on a pH of a fluid in contact with the medical device;
receiving a light signal from the coating in response to the projected light, the light signal including the light characteristic;
extracting the pH from the light signal; and
communicating the pH to the user.

12. The system according to claim 1, wherein the operations further include:
projecting the light into blood of the patient;
receiving a light signal from the blood in response to the projected light, the light signal including a light characteristic based on a blood oxygen level,
extracting the blood oxygen level from the light signal; and
communicating the blood oxygen level to the user.

13. The system according to claim 1, wherein the elongate medical device includes a catheter, a stylet, a probe, or a guidewire.
